(19) United States
(12) Patent Application Publication
(10) Pub. No.: US 2004/0077878 A1
(43) Pub. Date: Apr. 22, 2004

(54) Process for the preparation of tri-nitrogen containing heteroaryl-diamine derivatives useful as pharmaceutical agents and methods of producing pharmaceutical agents

(76) Inventor: Chunjian Liu, Pennington, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/683,096

(22) Filed: Oct. 10, 2003

Related U.S. Application Data
(60) Provisional application No. 60/418,102, filed on Oct. 11, 2002.

(51) Int. Cl. C07D 249/14

(52) U.S. Cl. 548/265.2

(57) ABSTRACT

This invention provides a process for producing at least one tri-nitrogen containing heteroaryl-diamine derivatives, which are useful as pharmaceutical agents and components, particularly as IMPDH inhibitors, comprising reacting an isothiocyanate with a hydrogencyanamide salt to produce an N-cyanothiourea salt, then reacting the salt with a hydrazine or amidine in the presence of a peptide-coupling reagent to provide the at least one tri-nitrogen containing heteroaryl-diamine derivative.

This application claims the benefit of U.S. Provisional Application No. 60/418,102, filed on Oct. 11, 2002, incorporated herein by reference.

### FIELD OF THE INVENTION

This invention relates to a process for producing tri-nitrogen containing heteroaryl-diamine derivatives, particularly 1,2,4-triazole-3,5-diamine and/or 1,3,5-triazine-2,4-diamine derivatives, useful as pharmaceutical agents and methods of making pharmaceutical agents comprising said process, particularly IMPDH inhibitors, neurokinin-1 (NK₁) receptor antagonists, and anti-hypertensive agents.

### BACKGROUND OF THE INVENTION

Tri-nitrogen containing heteroaryl-diamine derivatives are useful as pharmaceutical agents and as intermediates in the preparation of such agents. More particularly, 1,2,4-triazole-3,5-diamine and/or 1,3,5-triazine-2,4-diamine derivatives are useful as inhibitors of, and/or as intermediates in preparing inhibitors of, inosine monophosphate dehydrogenase (IMPDH), a key enzyme in the regulation of cell proliferation and differentiation. (See, e.g., Intern. application WO 00/25780, issued to Bristol-Myers Squibb Company.) Inhibitors of IMPDH are potentially useful in the treatment of solid organ transplant rejection, rheumatoid arthritis, psoriasis, autoimmune diseases, and other conditions. For example, mycophenolate mofetil, sold under the trade name CELLCEPT®, is a prodrug that liberates mycophenolic acid (“MPA”) in vivo and is approved for use in preventing acute renal allograft rejection following kidney transplantation. IMPDH inhibitors containing 1,2,4-triazolyl components are described in WO 00/25780.

Additionally, 1,2,4-triazole-3,5-diamine based compounds are described as effective antagonists of the neurokinin-1 (NK₁) receptor in Dunstan et al., Tetrahedron Lett., Vol. 39, (1998), at pp. 7983-7986. Neurokinin receptors are found in the nervous system, the circulatory system, and peripheral tissues of mammals. Consequently, they are involved in a variety of biological processes and mediate conditions or diseases related to inflammation and the central nervous system. See, e.g., U.S. Pat. No. 6,436,928 and U.S. patent application No. 2002/0038030.

1,2,4-Triazole-3,5-diamine derivatives and/or conversion products thereof are further disclosed as useful components of compounds effective as anti-hypertensive agents, bronchodilator agents, tachykinins antagonists for the treatment of central nervous system disorders, and anti-hyperproliferative agents. See, e.g., U.S. Pat. Nos. 6,172,077B1, 5,232,938, and 4,569,933, International applications nos. WO 01/74806A1, WO 01/56987 A1, and EP 1107962 A1, each incorporated herein by reference.

Hitherto-known processes for producing 1,2,4-triazole-3,5-diamine derivatives include the following:

(1) The process in which N-cyanoguanidine is coupled to a hydrazine (see Steck et al., J. Am. Chem. Soc., Vol. 80 [1958], at pp. 3929-3931, and Wu, J. Heterocyclic Chem., Vol. 14 [1977], at pp. 443-444);

(2) The process in which N-cyano-S-methylisothiourea is prepared from S,S′-dimethyl N-cyanodithioimidocarbonate, and then coupled to a hydrazine (see Wu, supra, and Reiter et al., J. Heterocyclic Chem., Vol. 23 [1986], at pp. 401-408); and

(3) The process in which diphenyl cyanocarbonimidate is treated with an aliphatic amine or aniline, and then hydrazine (see Webb et al., J. Heterocyclic Chem., Vol. 19 [1982], at pp. 1205-1206; Webb et al., J. Heterocyclic Chem., Vol. 24 [1987], pp. 275-278; Garratt et al., Tetrahedron, Vol. 49 [1993], at pp. 165-176; and Dunstan, et al. Tetrahedron Lett., Vol. 39 [1998], at pp. 7983-7986.)

Hitherto-known processes for producing 1,3,5-triazine-2,4-diamine derivatives include the following:

(1) The process in which 2,4-dichloro-6-aryl-1,3,5-triazine is prepared from cyanuric trichloride via a Grignard or Friedel-Crafts reaction, and then substituted with ammonia and amine (see Pitts et al., Bioorg. Med. Chem. Lett., 12 [2002], at pp. 2137-2140; Hirt et al., Helv. Chim. Acta., 33 [1950], at pp. 1365-1369; Migdal et al., U.S. Pat. No. 4,826,978 [1989]; and Chakrabarti, J. K. and Tupper, D. E., J. Heterocycl. Chem., 11 [1974], at pp. 417-421); and

(2) The process in which dicyandiamide is treated with an amine, and then an arylcarboxylate or carbonyl chloride (see Hajduk, P. J., J. Med. Chem., 42 [1999], at pp. 3852-3859; Brzozowski et al., Eur. J. Med. Chem., 37 [2002], at pp. 709-720; and Yuki et al., Bull. Chem. Soc. Jap., 43 [1970], at pp. 2130-2134.)

There remains a need for processes for preparing 1,2,4-triazole-3,5-diamine and/or 1,3,5-triazine-2,4-diamine derivatives demonstrating greater efficiency than prior art methods.

### SUMMARY OF THE INVENTION

According to one aspect of the invention, there is provided a process, preferably, a one-pot process, of producing at least one 1,2,4-triazole-3,5-diamine derivative comprising reacting an isothiocyanate with a hydrogencyanamide salt to produce an N-cyanothiourea salt, then reacting the N-cyanothiourea salt with a hydrazine in the presence of a peptide-coupling reagent to provide the at least one 1,2,4-triazole-3,5-diamine derivative.

Similarly, according to another aspect of the invention, there is provided a process, preferably, a one-pot process, of producing a 1,3,5-triazine-2,4-diamine derivative comprising reacting an isothiocyanate with a hydrogencyanamide salt to produce an N-cyanothiourea salt, then reacting the N-cyanothiourea salt with an amidine in the presence of a peptide-coupling reagent to provide the 1,3,5-triazine-2,4-diamine derivative.

According to another aspect of the invention, there is provided a process for producing tri-nitrogen containing heteroaryl-diamine derivatives of formula (I):

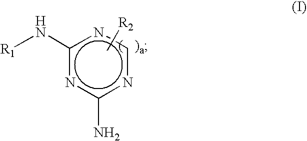

in which a is 0 or 1 and R₁ and R₂ are independently selected from alkyl, substituted alkyl, cycloalkyl, heterocyclo, aryl, and heteroaryl,

which comprises:

(a) reacting an isothiocyanate (R₁NCS) with a hydrogencyanamide salt in an organic solvent to produce an N-cyanothiourea salt;

(b) reacting the N-cyanothiourea salt with (i) a hydrazine (R₂NHNH₂), an amidine (R₂C(NH)NH₂), a salt of a hydrazine with one or more equivalents of a base, or a salt of an amidine with one or more equivalents of a base, and (ii) in the presence of a peptide-coupling reagent, to afford the tri-nitrogen containing heteroaryl-diamine derivatives of formula (I).

In a preferred embodiment of the present invention, there is provided a process for producing 1,2,4-triazole-3,5-diamine derivatives of the formula (A) and/or (B):

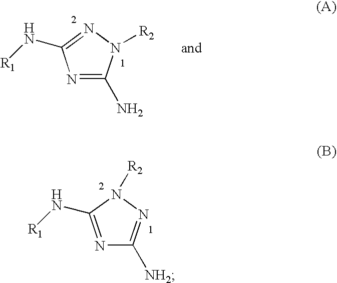

in which R₁ and R₂ are independently selected from alkyl, substituted alkyl, cycloalkyl, heterocyclo, aryl, and heteroaryl,

which comprises:

(a) reacting an isothiocyanate (R₁NCS) with a hydrogencyanamide salt in an organic solvent to produce an N-cyanothiourea salt;

(b) reacting the N-cyanothiourea salt with (i) a hydrazine (R₂NHNH₂), or a salt of a hydrazine with one or more equivalents of a base, and (ii) in the presence of a peptide-coupling reagent, to afford the 1,2,4-triazole-3,5-diamine derivatives of the formula (A) and/or (B).

In another preferred embodiment of the present invention, there is provided a process for producing 1,3,5-triazine-2,4-diamine derivatives of formula (C):

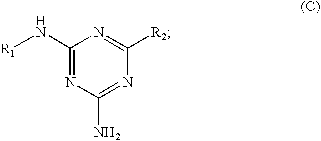

in which R₁ and R₂ are independently selected from alkyl, substituted alkyl, cycloalkyl, heterocyclo, aryl, and heteroaryl,

which comprises:

(a) reacting an isothiocyanate (R₁NCS) with a hydrogencyanamide salt in an organic solvent to produce an N-cyanothiourea salt;

(b) reacting the N-cyanothiourea salt with (i) an amidine (R₂C(NH)NH₂), or a salt of an amidine with one or more equivalents of a base, and (ii) in the presence of a peptide-coupling reagent, to afford the 1,3,5-triazine-2,4-diamine derivatives of the formula (C).

According to another aspect of the invention, there is provided a process for producing a pharmaceutical agent comprising steps (a) and (b), as recited above, and also a step of coupling a compound of formulae (I), (A), (B) and/or (C), with a pharmacological core component to produce an active pharmacological agent.

According to yet another aspect of the invention, there is provided a process for producing tri-nitrogen containing heteroaryl-diamine derivatives of the formulae (I), (A), (B) and (C), as immediately defined above, wherein R₁ is a pharmacological core component (X) as defined herein.

### DETAILED DESCRIPTION OF THE INVENTION

The following are definitions of terms used in this specification. The initial definition provided for a group or term herein applies to that group or term throughout the present specification, individually or as part of another group, unless otherwise indicated.

The term “alkyl” refers to straight or branched chain hydrocarbon groups having 1 to 12 carbon atoms, preferably 1 to 8 carbon atoms. Lower alkyl groups, that is, alkyl groups of 1 to 4 carbon atoms, are most preferred.

The term “substituted alkyl” refers to an alkyl group as defined above having one, two, or three substituents selected from the group consisting of halogen, trifluoromethyl, alkenyl, alkynyl, nitro, cyano, keto (═O), ORₐ, SRₐ, NRₐR_{b}, NRₐSO₂, NRₐSO₂R_{c}, SO₂R_{c}, SO₂NRₐR_{b}, CO₂Rₐ, C(═O)Rₐ, C(═O)NRₐR_{b}, OC(═O)Rₐ, —OC(═O)NRₐR_{b}, NRₐC(═O)R_{b}, NRₐCO₂R_{b}, ═N—OH, ═N—O-alkyl, aryl, heteroaryl, heterocyclo and cycloalkyl, wherein Rₐ and R_{b} are selected from hydrogen, alkyl, alkenyl, cycloalkyl, heterocyclo, aryl, and heteroaryl, and R_{c} is alkyl, alkenyl, cycloalkyl, heterocyclo, aryl, or heteroaryl. When a substituted alkyl includes an aryl, heterocyclo, heteroaryl, or cycloalkyl substituent, said ringed systems are as defined below and thus may in turn have zero to three substituents (preferably 0-2 substituents), also as defined below. When either Rₐ or R_{b} is an alkyl, said alkyl may optionally be substituted with 1-2 of halogen, trifluoromethyl, alkenyl, alkynyl, nitro, cyano, keto (═O), OH, O(alkyl), phenyloxy, benzyloxy, SH, S(alkyl), NH₂, NH(alkyl), N(alkyl)₂, NHSO₂, NHSO₂(alkyl), SO₂H, SO₂(alkyl), SO₂NH₂, SO₂NH(alkyl), CO₂H, CO₂(alkyl), C(═O)H, C(═O)alkyl, C(═O)NH₂, C(═O)NH(alkyl), C(═O)N(alkyl)₂, OC(═O)alkyl, —OC(═O)NH₂, —OC(═O)NH(alkyl), NHC(═O)alkyl, and/or NHCO₂(alkyl).

“Alkyl” when used in conjunction with another group such as in arylalkyl refers to a substituted alkyl in which at least one of the substituents is the specifically-named group. For example, the term arylalkyl includes benzyl, or any other straight or branched chain alkyl having at least one aryl group attached at any point of the alkyl chain.

The term “thioalkyl” refers to an alkyl or substituted alkyl group as defined above bonded through a sulfur (—S—) atom. For example, the term “thioalkyl” includes the groups —S—CH₂aryl, —S(CH₂)ₙ—CH₃, etc.

When a subscript is used as in C₁₋₈alkyl, the subscript refers to the number of carbon atoms the group may contain. When zero is used in a subscript, this denotes a bond, e.g., C₀₋₄alkyl refers to a bond or an alkyl of 1 to 4 carbon atoms.

The term “cycloalkyl” refers to fully saturated and partially unsaturated hydrocarbon rings of 3 to 9, preferably 3 to 7 carbon atoms. The term “cycloalkyl” includes such rings having zero to three substituents (preferably 0-2 substituents), selected from the group consisting of halogen, alkyl, substituted alkyl (e.g., trifluoromethyl), alkenyl, substituted alkenyl, alkynyl, nitro, cyano, keto, ORₐ, SRₐ NRₐR_{b}NRₐSO₂, NRₐSO₂R_{c}, C(═O)H, acyl, —CO₂H, alkoxycarbonyl, carbamyl, sulfonyl, sulfonamidyl, —OC(═O)Rₐ, ═N—OH, ═N—O-alkyl, aryl, heteroaryl, heterocyclo, a 4 to 7 membered carbocyclic ring, and a five or six membered ketal, e.g., 1,3-dioxolane or 1,3-dioxane, wherein Rₐ, R_{b}, and R_{c} are defined as above. The term “cycloalkyl” also includes such rings having a phenyl ring fused thereto or having a carbon-carbon bridge of 3 to 4 carbon atoms. Additionally, when a cycloalkyl is substituted with a further ring, i.e., aryl, arylalkyl, heteroaryl, heteroarylalkyl, heterocyclo, heterocycloalkyl, cycloalkylalkyl, or a further cycloalkyl ring, such ring in turn may be substituted with one to two of C₀₋₄alkyl optionally substituted with halogen, trifluoromethyl, alkenyl, alkynyl, nitro, cyano, keto (═O), OH, O(alkyl), phenyloxy, benzyloxy, SH, S(alkyl), NH₂, NH(alkyl), N(alkyl)₂, NHSO₂, NHSO₂(alkyl), SO₂H, SO₂(alkyl), SO₂NH₂, SO₂NH(alkyl), CO₂H, CO₂(alkyl), C(═O)H, C(═O)alkyl, C(═O)NH₂, C(═O)NH(alkyl), C(═O)N(alkyl)₂, OC(═O)alkyl, —OC(═O)NH₂, —OC(═O)NH(alkyl), NHC(═O)alkyl, and NHCO₂(alkyl).

The term “halo” or “halogen” refers to chloro, bromo, fluoro and iodo.

The term “hydrazine” as used herein means a compound having the formula R₂NHNH₂, wherein R₂ is selected from a hydrogen, alkyl, substituted alkyl, aryl, heteroaryl, heterocyclo, or cycloalkyl group, as defined herein, and any pharmacological core component, X, as defined herein.

The term “amidine” as used herein means a compound having the formula R₂C(NH)NH₂, wherein R₂ is selected from a hydrogen, alkyl, substituted alkyl, aryl, heteroaryl, heterocyclo, or cycloalkyl group, as defined herein, and any pharmacological core component, X, as defined herein.

The term “isothiocyanate” as used herein means a compound having the formula R₁NCS, wherein R₁ includes an alkyl, substituted alkyl, aryl, heteroaryl, heterocyclo, or cycloalkyl group, as defined herein, and any pharmacological core component, X, as defined herein.

The term “peptide-coupling reagent” as used herein means a reagent used to couple a carboxylic acid and an amine or an aniline to form an amide bond. It may include a coupling additive, such as CDI, HOBt, HOAt, HODhbt, HOSu, or NEPIS, used in combination with another coupling reagent to speed up the coupling process and inhibit side reactions. Particular peptide-coupling reagents may include DCC, EDC, BBC, BDMP, BOMI, HATU, HAPyU, HBTU, TAPipU, AOP, BDP, BOP, PyAOP, PyBOP, TDBTU, TNTU, TPTU, TSTU, BEMT, BOP-Cl, BroP, BTFFH, CIP, EDPBT, Dpp-Cl, EEDQ, FDPP, HOTT-PF6, TOTT-BF4, PyBrop, PyClop, and TFFH. See “Peptide Coupling Reagents: Names, Acronyms and References,” Albany Molecular Research, Inc., Technical Reports, Vol. 4, No. 1.

The term “aryl” refers to phenyl, biphenyl, 1-naphthyl, and 2-naphthyl, with phenyl being preferred. The term “aryl” includes such rings having zero to three substituents (preferably 0-2 substituents), selected from the group consisting of halo, alkyl, substituted alkyl (e.g., trifluoromethyl), alkenyl, substituted alkenyl, alkynyl, nitro, cyano, ORₐ, SRₐ NRₐR_{b} NRₐSO₂, NRₐSO₂R_{c}, C(═O)H, acyl, —CO₂H, alkoxycarbonyl, carbamyl, sulfonyl, sulfonamidyl, —OC(═O)Rₐ, heteroaryl, heterocyclo, cycloalkyl, phenyl, benzyl, napthyl, including phenylethyl, phenyloxy, and phenylthio, wherein Rₐ, R_{b}, and R_{c} are defined as above. Additionally, two substituents attached to an aryl, particularly a phenyl group, may join to form a further ring such as a fused or spiro-ring, e.g., cyclopentyl or cyclohexyl or fused heterocycle or heteroaryl. When an aryl is substituted with a further ring, such ring in turn may be substituted with one to two of C₀₋₄alkyl optionally substituted with halogen, trifluoromethyl, alkenyl, alkynyl, nitro, cyano, keto (═O), OH, O(alkyl), phenyloxy, benzyloxy, SH, S(alkyl), NH₂, NH(alkyl), N(alkyl)₂, NHSO₂, NHSO₂(alkyl), SO₂H, SO₂(alkyl), SO₂NH₂, SO₂NH(alkyl), CO₂H, CO₂(alkyl), C(═O)H, C(═O)alkyl, C(═O)NH₂, C(═O)NH(alkyl), C(═O)N(alkyl)₂, OC(═O)alkyl, —OC(═O)NH₂, —OC(═O)NH(alkyl), NHC(═O)alkyl, and NHCO₂(alkyl).

The term “heterocyclo” refers to substituted and unsubstituted non-aromatic 3 to 7 membered monocyclic groups, 7 to 11 membered bicyclic groups, and 10 to 15 membered tricyclic groups, in which at least one of the rings has at least one heteroatom (O, S or N). Each ring of the heterocyclo group containing a heteroatom can contain one or two oxygen or sulfur atoms and/or from one to four nitrogen atoms provided that the total number of heteroatoms in each ring is four or less, and further provided that the ring contains at least one carbon atom. The fused rings completing bicyclic and tricyclic groups may contain only carbon atoms and may be saturated, partially saturated, or unsaturated. The nitrogen and sulfur atoms may optionally be oxidized and the nitrogen atoms may optionally be quaternized. The heterocyclo group may be attached at any available nitrogen or carbon atom. The heterocyclo ring may contain zero to three substituents (preferably 0-2 substituents), selected from the group consisting of halo, alkyl, substituted alkyl (e.g., trifluoromethyl), alkenyl, substituted alkenyl, alkynyl, nitro, cyano, keto, ORₐ, SRₐ NRₐR_{b} NRₐSO₂, NRₐSO₂R_{c}, C(═O)H, acyl, —CO₂H, alkoxycarbonyl, carbamyl, sulfonyl, sulfonamidyl, —OC(═O)Rₐ, ═N—OH, ═N—O-alkyl, aryl, heteroaryl, cycloalkyl, a five or six membered ketal, e.g., 1,3-dioxolane or 1,3-dioxane, or a monocyclic 4 to 7 membered non-aromatic ring having one to four heteroatoms, wherein Rₐ, R_{b}, and R_{c} are defined as above. The term “heterocyclo” also includes such rings having a phenyl ring fused thereto or having a carbon-carbon bridge of 3 to 4 carbon atoms. Additionally, when a heterocyclo is substituted with a further ring, i.e., aryl, arylalkyl, heteroaryl, heteroarylalkyl, cycloalkyl, cycloalkylalkyl, heterocycloalkyl, or a further heterocyclo ring, such ring in turn may be substituted with one to two of C₀₋₄alkyl optionally substituted with halogen, trifluoromethyl, alkenyl, alkynyl, nitro, cyano, keto (═O), OH, O(alkyl), phenyloxy, benzyloxy, SH, S(alkyl), NH₂, NH(alkyl), N(alkyl)₂, NHSO₂, NHSO₂(alkyl), SO₂H, SO₂(alkyl), SO₂NH₂, SO₂NH(alkyl), CO₂H, CO₂(alkyl), C(═O)H, C(═O)alkyl, C(═O)NH₂, C(═O)NH(alkyl), C(═O)N(alkyl)₂, OC(═O)alkyl, —OC(═O)NH₂, —OC(═O)NH(alkyl), NHC(═O)alkyl, and NHCO₂(alkyl).

Exemplary monocyclic groups include azetidinyl, pyrrolidinyl, oxetanyl, imidazolinyl, oxazolidinyl, isoxazolinyl, thiazolidinyl, isothiazolidinyl, tetrahydrofuranyl, piperidinyl, piperazinyl, 2-oxopiperazinyl, 2-oxopiperidinyl, 2-oxopyrrolodinyl, 2-oxoazepinyl, azepinyl, 4-piperidonyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl and the like. Exemplary bicyclic heterocyclo groups include quinuclidinyl.

The term “heteroaryl” refers to substituted and unsubstituted aromatic 5 to 7 membered monocyclic groups, 9 or 10 membered bicyclic groups, and 11 to 14 membered tricyclic groups which have at least one heteroatom (O, S or N) in at least one of the rings. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic and tricyclic groups may contain only carbon atoms and may be saturated, partially saturated, or unsaturated. The nitrogen and sulfur atoms may optionally be oxidized and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. The heteroaryl ring system may contain zero to three substituents (preferably 0-2 substituents), selected from the group consisting of halo, alkyl, substituted alkyl (e.g., trifluoromethyl), alkenyl, substituted alkenyl, alkynyl, nitro, cyano, ORₐ, SRₐ NRₐR_{b} NRₐSO₂, NRₐSO₂R_{c}, C(═O)H, acyl, —CO₂H, alkoxycarbonyl, carbamyl, sulfonyl, sulfonamidyl, —OC(═O)Rₐ, heterocyclo, cycloalkyl, aryl, or a monocyclic 4 to 7 membered aromatic ring having one to four heteroatoms, wherein Rₐ, R_{b}, and R_{c} are defined as above. Additionally, when a heteroaryl is substituted with a further ring, i.e., aryl, arylalkyl, heterocyclo, heterocycloalkyl, cycloalkyl, cycloalkylalkyl, heteroarylalkyl, or a further heteroaryl ring, such ring in turn may be substituted with one to two of C₀₋₄alkyl optionally substituted with halogen, trifluoromethyl, alkenyl, alkynyl, nitro, cyano, keto (═O), OH, O(alkyl), phenyloxy, benzyloxy, SH, S(alkyl), NH₂, NH(alkyl), N(alkyl)₂, NHSO₂, NHSO₂(alkyl), SO₂H, SO₂(alkyl), SO₂NH₂, SO₂NH(alkyl), CO₂H, CO₂(alkyl), C(═O)H, C(═O)alkyl, C(═O)NH₂, C(═O)NH(alkyl), C(═O)N(alkyl)₂, OC(═O)alkyl, —OC(═O)NH₂, —OC(═O)NH(alkyl), NHC(═O)alkyl, and NHCO₂(alkyl).

Exemplary monocyclic heteroaryl groups include pyrrolyl, pyrazolyl, pyrazolinyl, imidazolyl, oxazolyl, isoxazolyl, thiazolyl (i.e.,

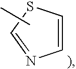

thiadiazolyl, isothiazolyl, furanyl, thienyl, oxadiazolyl, pyridyl, pyrazinyl, pyrimidinyl, pyridazinyl, triazinyl and the like.

Exemplary bicyclic heteroaryl groups include indolyl, benzothiazolyl, benzodioxolyl, benzoxaxolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, pyrrolopyridyl, furopyridinyl, dihydroisoindolyl, tetrahydroquinolinyl and the like.

Exemplary tricyclic heteroaryl groups include carbazolyl, benzidolyl, phenanthrollinyl, acridinyl, phenanthridinyl, xanthenyl and the like.

The term “pharmacological core component” means a component (X) which, when coupled to a triazolyl or triazinyl group via a nitrogen atom (X—NH-triazolyl or X—NH-triazinyl), has a measurable level of activity for agonizing or antagonizing an enzyme or receptor known to be involved in one or more biological functions in a mammal. For example, U.S. Pat. No. 5,232,938 to Stemp et al., describes compounds useful as potassium channel activators having the formula,

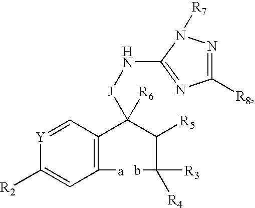

wherein the group R₈ may be amino. Accordingly, as defined herein the term “pharmacological core component” includes the component having the formula,

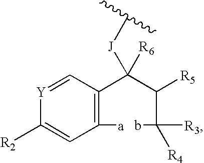

wherein J, R₂, R₃, R₄, R₅, R₆ and a and b are as described in U.S. Pat. No. 5,232,938, for preparation of compounds effective as potassium channel activators. As a further illustration, Intern. application WO 00/25780 describes compounds having the formula

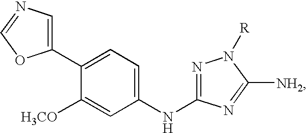

as inhibitors of IMPDH enzyme, e.g., where the R groups may be selected from NH₂, phenyl, pyridyl, NH(C(═O)(alkyl), and so forth. The term “pharmacological core component” (or X) thus includes the component having the formula,

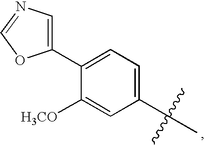

e.g., for preparation of compounds effective as inhibitors of IMPDH. As yet a further illustration, U.S. Pat. No. 4,569,933 describes compounds having the formula

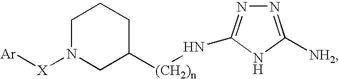

as anti-hypertensive agents. Thus, “pharmacological core component” includes the group

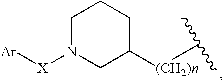

wherein Ar, X, and n are as described in U.S. Pat. No. 4,569,933. One skilled in the field will appreciate that there are many applications in which a 1,2,4-triazole-3,5-diamine and/or 1,3,5-triazine-2,4-diamine may be coupled to a pharmacological core or scaffold to produce a pharmacologically active agent. The instant invention encompasses such applications that include use of the efficient process, preferably, a one-pot process, described herein of producing 1,2,4-triazole-3,5-diamine and/or 1,3,5-triazine-2,4-diamine via reaction of an isothiocyanate with a hydrogencyanamide salt, followed by reaction with a hydrazine and/or amidine in the presence of a peptide-coupling reagent.

Throughout the specification, groups and substituents thereof may be chosen by one skilled in the field to provide stable moieties and compounds.

The compounds of formulae (I), (A), (B) and (C) may form salts, and processes including use or preparation of such salts are encompassed within the scope of this invention. The term “salt(s)”, as employed herein, denotes acidic and/or basic salts formed with inorganic and/or organic acids and bases. In addition, when a compound of formulae (I), (A), (B) or (C) contains both a basic moiety, such as, but not limited to, an amine or a pyridine or imidazole ring, and an acidic moiety, such as, but not limited to, a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation. Salts of the compounds of the formulae (I), (A), (B) or (C) may be formed, for example, by reacting a compound of the formulae (I), (A), (B) or (C) with an amount of acid or base, such as an equivalent amount, in a medium such as one in which the salt precipitates or in an aqueous medium followed by lyophilization.

The compounds of formulae (I), (A), (B) and (C) may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Compounds of the formulae (I), (A), (B) and (C), and salts thereof, may exist in their tautomeric form (for example, as an amide or imino ether). Processes including the preparation of such tautomeric forms are contemplated herein as part of the present invention.

In performing the processes according to the present invention, racemic mixtures of stereoisomers of compounds of formulae (I), (A), (B) and (C) may be prepared. Stereoisomers may exist due to asymmetric carbons, including enantiomeric forms (which may exist even in the absence of asymmetric carbons), and diastereomeric forms. Additionally, the processes according to the invention may be used to prepare individual stereoisomers of the compounds of formulae (I), (A), (B) and (C), for example, substantially free of other isomers, e.g., purification steps may be employed. The chiral centers of the compounds prepared according to the present invention can have the S or R configuration as defined by the IUPAC 1974 Recommendations.

Processes for producing a 1,2,4-triazole-3,5-diamine and/or 1,3,5-triazine-2,4-diamine derivative in accordance with the present invention are now described.

According to one aspect of the invention, a 1,2,4-triazole-3,5-diamine derivative is prepared by (a) adding a hydrogen cyanamide salt to a solution of an appropriately-substituted isothiocyanate (R₁NCS) in an organic solvent, and (b) reacting the resulting mixture of step (a) with an appropriately-substituted hydrazine (R₂NHNH₂) in the presence of a peptide-coupling reagent, such as EDC, to provide the 1,2,4-triazole-3,5-diamine derivative. Exemplary hydrogen cyanamide salts include salts of the formula M(NHCN)ₙ, wherein M is Li, Na, K, Mg, Ca or Ba, and n is 1 or 2. Alternative to the use of hydrazine, a hydrazine hydrochloride salt (or other salt of hydrazine) can be used together with a base, such as triethylamine. This process may be efficiently carried out in one pot, e.g.:

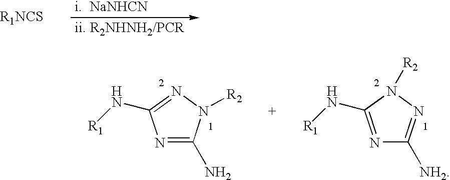

According to another aspect of the invention, a 1,3,5-triazine-2,4-diamine derivative can be prepared in the manner described in steps (a) and (b) above with the exception that an appropriately substituted amidine (R₂C(NH)NH₂) rather than hydrazine is used. The hydrogen cyanamide salts, isothiocyanates, and peptide-coupling reagents set forth above may also be used in this embodiment. Similar to the process described above, alternative to the use of amidine, an amidine hydrochloride salt (or other salt of amidine) can be used together with a base. This process may also be efficiently carried out in one pot, e.g.:

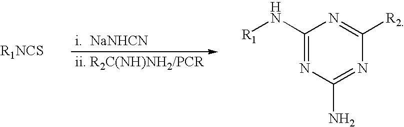

The inventive process is advantageous in that it can be performed in one pot, allows for the preparation of a broad scope of substituted triazolyl and/or triazinyl compounds, and proceeds via use of readily-available starting materials. Aromatic or aliphatic isothiocyanates, hydrazine and/or amidine compounds can be used, with aromatic isothiocyanates typically producing yields of greater than 40%, preferably greater than 65%, more preferably greater than 70%. The isothiocyanates, hydrazines and amidines can be substituted with electron donating or electron withdrawing groups, and even highly sterically-hindered hydrazines do not have a significant affect on the reaction.

In carrying out the process, the mixture of isothiocyanate and hydrogen cyanamide salt described in step (a) may be stirred at a first elevated temperature for a period of time and then cooled to ambient temperature before the hydrazine or amidine and peptide-coupling reagent, e.g., EDC, are added. Once the hydrazine or amidine and coupling component are added, the reaction may be heated (again) to a second elevated temperature for a second time period, with stirring, and then cooled to ambient temperature.

One skilled in the field can select appropriate substituents for the isothiocyanate, hydrazine and amidine considering the compounds sought to be produced, as well as appropriate reaction temperatures and solvents. However, the first and second elevated temperatures for the reaction are preferably selected from a temperature in the range of −10° C. to 120° C., more preferably from a temperature in the range of 20° C.-120° C., and even more preferably in the range of 40° C.-80° C. The ambient temperature is preferably −10° C. to 30° C., more preferably 15° C.-25° C. Preferably, the isothiocyanate is substituted with a group selected from (i.e., the group R₁ may be selected from) alkyl, substituted alkyl, cycloalkyl, heterocyclo, aryl, or heteroaryl, as defined herein. However, any use of the inventive process is contemplated as included within the scope of the invention, e.g., where the isothiocyanate is bonded to a pharmacological core component (X). More preferred are processes where the group R₁ is an optionally-substituted phenyl group. The hydrazine and/or amidine also may be aromatic or aliphatic. For example, the hydrazine and/or amidine may be substituted with (i.e., the group R₂ may be selected from) alkyl, substituted alkyl, cycloalkyl, heterocyclo, aryl, or heteroaryl. More preferred hydrazine and/or amidine (R₂) groups include optionally-substituted alkyl, cycloalkyl (e.g., cyclohexyl), and phenyl groups.

The reaction time for each of the first and second steps is preferably selected from a time in the range of 5 minutes to 48 hours, more preferably in the range of 30 minutes to 24 hours (for each step).

The solvent used to run the reaction may be any appropriate organic solvent. It may be selected from, for example, aprotic polar solvents such as DMF, DMA, DMSO, dimethylpropyleneurea, N-methylpyrrolidone, and hexamethylphosphoric triamide; ether solvents such as diethyl ether, THF, 1,4-dioxane, methyl t-butyl ether, dimethoxymethane, and ethylene glycol dimethyl ether; alcohol solvents such as MeOH, EtOH, propanol, isopropanol, n-butylalcohol and t-butyl alcohol; and halogen-containing solvents such as methylene chloride, chloroform, carbon tetrachloride, and 1,2-dichloroethane. These solvents may be used each alone, or two or more of the solvents may be used in a suitable combination. Preferred, among the above-mentioned solvents, are aprotic polar solvents such as DMF, DMA, DMSO, and the like, and ether solvents such as THF, 1,4-dioxane, dimethoxymethane, and/or ethylene glycol dimethyl ether.

An aftertreatment may be performed which may include work-up steps known in the field for recovery of the reaction product from a reaction mixture. A typical procedure may comprise diluting the reaction mixture with an organic solvent, such as ethyl acetate, methylene chloride, diethyl ether, toluene, or the like, or a mixture of two or more of these organic solvents, and then washing the organic layer with water and/or an aqueous inorganic salt solution, such as 10% lithium chloride, one or more times. The organic layer may be dried over a dehydrating agent, such as anhydrous MgSO₄ or Na₂SO₄, and then concentrated under reduced pressure. The product thus obtained may be purified using techniques known to one skilled in the field, such as crystallization, column chromatography and/or the like, to further enhance its purity.

Compounds of formulae (I), (A), (B) and (C) may be purified and/or may be further reacted to produce a desired pharmacological agent. For example, the 1,2,4-triazole-3,5-diamine and/or 1,3,5-triazine-2,4-diamine derivative produced from steps (a) and (b), may (with or without further isolation or purification), be coupled to a pharmacological core component to produce a desired pharmacological agent, and/or the 3,5- and/or 2,4-amino groups and/or the groups R₁, R₂ may be replaced with or converted to an alternative group imparting biological activity or enhanced biological activity to the compound, applying techniques known in the field. For example, the 5-amino group of the 1,2,4-triazole-3,5-diamine derivative can be coupled with an acid chloride in the presence of a base to form an amide. It can also be alkylated through reductive amination, e.g., to form —NHCH₂CH₃ by reacting with an aldehyde in the presence of sodium triacetoxyborohydride.

### Abbreviations

The following abbreviations are employed in the Examples and elsewhere herein, for ease of reference:

AOP=O-(7-azabenzotriazol-1-yl)-tris(dimethylamino)phosphonium hexafluorophosphate

aq.=aqueous

BBC=1-benzotriazol-1-yloxy-bis(pyrrolidino)uronium hexafluorophosphate

BDMP=5-(1H-benzotriazol-1-yloxy)-3,4-dihydro-1-methyl 2H-pyrrolium hexachloroanitimonate

BDP=benzotriazol-1-yl diethyl phosphate

BEMT=2-bromo-3-ethyl-4-methyl thiazolium tetrafluoroborate

BOMI=benzotriazol- 1 -yloxy-N,N-dimethylmethaniminium hexachloroantimonate

BOP=benzotriazol-1-yloxy-tris(dimethylamino)phosphonium hexafluorophosphate

BOP-Cl=bis(2-oxo-3-oxazolidinyl)phosphinic chloride

BroP=bromotris(dimethylamino)phosphonium hexafluorophosphate

BTFFH=bis(tetramethylenefluoroformamidinium)hexafluorophosphate

CDI=carbonyldiimidazole

CIP=2-chloro-1,3-dimethylimidazolidinium hexafluorophosphate

DCC=1,3-dicyclohexylcarbodiimide

DCE=1,2-dichloroethane

DCM=dichloromethane

DEPBT=3-(diethoxyphosphoryloxy)-1,2,3-benzotriazin-4(3H)-one

DMA=N,N-dimethylacetimide

DMF=N,N-dimethylformamide

DMSO=dimethylsulfoxide

Dpp-Cl=diphenylphosphinic chloride

EDC=1-(3-dimethylaminopropyl)-3-ethylcarbodiimide hydrochloride

EEDQ=2-ethoxy-1-ethoxycarbonyl-1,2-dihydroquinoline

EtOAc=ethyl acetate

EtOH=ethanol

FDPP=pentafluorophenyl diphenylphosphinate

g=gram(s)

h or hr=hour(s)

HAPyU=O-(7-azabenzotriazol-1-yl)-1,1,3,3-bis(tetramethylene)uronium hexafluorophosphate

HATU=O-(7-azabenzotriazol-1-yl)-1,1,3,3-tetramethyluronium hexafluorophosphate

HBTU=O-(benzotriazol-1-yl)-1,1,3,3-tetramethyluronium hexafluorophosphate

HOAT=1-hydroxy-7-azabenzotriazole

HOBt=1-hydroxybenzotriazole

HODhbt=3-hydroxy-3,4-dihydro-4-oxo-1,2,3-benzotriazine

HOSu=hydroxysuccinimide

HOTT=S-(1-oxido-2-pyridinyl)-1,1,3,3-tetramethylthiouronium hexafluorophosphate

IMPDH=inosine monophosphate dehydrogenase

L=liter

μL=microliter

LC/MS=high performance liquid chromatography/mass spectrometry

MeOH=methanol

mg=milligram(s)

min=minute(s)

MgSO₄=magnesium sulfate

mL=milliliter

MS or Mass Spec=mass spectrometry

mol=mole(s)

mp=melting point

Na₂SO₄=sodium sulfate

NEPIS=N-ethyl-5-phenylisoxazolium-3′-sulfonate

NK₁=neurokinin-1

PCR=peptide-coupling reagent

PyAOP=7-azobenzotriazolyoxytris(pyrrolidino)phosphonium hexafluorophosphate

PyBOP=1-benzotriazolyoxytris(pyrrolidino)phosphonium hexafluorophosphate

PyBroP=bromotris(pyrrolydino)phophonium hexafluorophosphate

PyCloP=chlorotris(pyrrolydino)phophonium hexafluorophosphate

rt=room temperature

sat or sat'd=saturated

TAPipU=O-(7-azabenzotriazol-1-yl)-1,1,3,3-bis(pentamethylene)uronium tetrafluoroborate

TDBTU=2-(3,4-dihydro-4-oxo-1,2,3-benzotriazin-3-yl)-1,1,3,3-tetramethyluronium tetrafluoroborate

TEA=triethylamine or Et₃N

TFA=trifluoroacetic acid

TFFH=tetramethylfluoroformamidinium hexafluorophosphate

THF=tetrahydrofuran

TNTU=2-(5-norbornene-2,3-dicarboximido)-1,1,3,3-tetramethyluronium tetrafluoroborate

TOTT=S-(1-oxido-2-pyridinyl)-1,1,3,3-tetramethylthiouronium tetrafluoroborate

TPTU=2-(2-oxo-1(2H)-pyridyl-1,1,3,3-tetramethyluronium tetrafluoroborate

TSTU=2-succinimido-1,1,3,3-tetramethyluronium tetrafluoroborate

### EXAMPLES

The following examples illustrate the present invention in further detail without defining its metes and bounds.

### Example 1

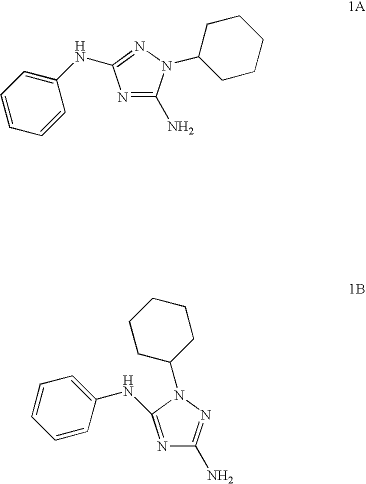

To a solution of phenyl isothiocyanate (0.276 g, 2.00 mmol) in dry DMF was added sodium hydrogencyanamide (0.137 g, 2.10 mmol) at room temperature in one portion. The mixture was stirred at 60° C. for 1 hour and then cooled to room temperature. Triethylamine (0.50 mL, 3.59 mmol), cyclohexylhydrazine hydrochloride (0.452 g, 3.00 mmol), and EDC (0.479 g, 2.50 mmol) were added. The mixture was re-heated at 60° C., stirred for an additional hour, and then cooled to room temperature. The mixture was diluted with ethyl acetate (100 mL), washed with water (3×25 mL) and 10% lithium chloride solution (3×30 mL). The organic solution was over anhydrous MgSO₄. A mixture of 1A and 1B (0.424 g, 82% yield, ratio of 1A/1B=3/1) was isolated as a white solid by silica gel chromatography (5% methanol/chloroform). Pure 1A and 1B were obtained by preparative HPLC.

### Examples 2-10

Compounds having formula (A) and (B), wherein R₁ and R₂ are shown in Table 1, were prepared using the method described in Example 1, starting with the corresponding isothiocyanate and hydrazine.

TABLE 1

| | | | Total | Ratio of | Mass | HPLC retention |
|---|---|---|---|---|---|---|
| Ex. | R₁ | R₂ | yield (%) | A to B | (M + H)⁺ | time (min) |
| 2 | 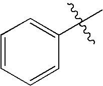 | 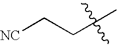 | 72 | 3.6:1 | 229 | 2A: 1.84 |
| | | | | | | 2B: 1.10 |
| 3 | 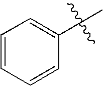 | 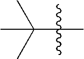 | 90 | 20:1 | 232 | 3A: 2.74 |
| | | | | | | 3B: 1.88 |
| 4 | 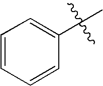 | 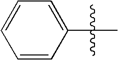 | 73 | 19:1 | 252 | 4A: 3.29 |
| | | | | | | 4B: 2.59 |
| 5 | 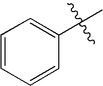 | 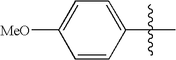 | 79 | 12:1 | 282 | 5A: 2.73 |
| | | | | | | 5B: 2.23 |
| 6 | 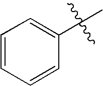 | 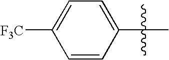 | 70 | 100:0 | 320 | 6A: 3.38 |
| 7 | 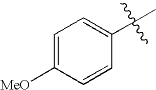 | 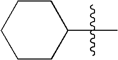 | 85 | 3.9:1 | 288 | 7A: 2.87 |
| | | | | | | 7B: 2.32 |
| 8 | 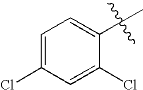 | 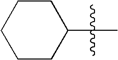 | 90 | 4.0:1 | 326 | 8A: 3.57 |
| | | | | | | 8B: 2.96 |
| 9 | 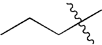 | 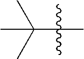 | 52 | 14:1 | 198 | 9A: 2.21 |
| | | | | | | 9B 1.42 |
| 10 | 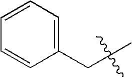 | 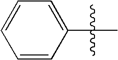 | 42 | 20:1 | 266 | 10A: 2.58 |
| | | | | | | 10B: 2.11 |

HPLC condition:
Column: YMC ODSA 5u C18 4.6 × 50 mm.
Solvent: solvent A = 10% MeOH/90% water/0.1% THF, and solvent B = 90%
MeOH/10% water/0.1% THF.
Method: 4 min gradient, start 0% B, final 100% B.

### Example 13

Compound 13 of Example 13, below, can be prepared using the method described in Example 1, starting with the corresponding isothiocyanate and hydrazine.

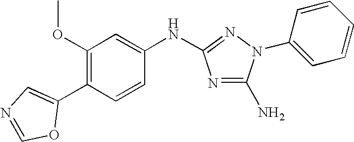

### Example 14

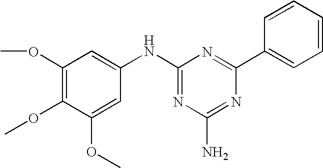

To a solution of 3,4,5-trimethoxyphenyl isothiocyanate (0.230 g, 98%, 1.00 mmol) in dry DMF was added sodium hydrogencyanamide (0.0686 g, 1.05 mmol) at room temperature in one portion. The mixture was heated at 60° C. for one hour before triethylamine (0.31 mL, 2.22 mmol), benzamidine hydrochloride (0.235 g, 1.50 mmol), and EDC (0.249 g, 1.30 mmol) were added at room temperature. The mixture was stirred at room temperature for 30 minutes and then heated to 75° C. where it stirred for two hours. After this period, the reaction mixture was cooled to room temperature, diluted with ethyl acetate (60 mL), and washed with water (3×20 mL) and a 10% lithium chloride solution (20 mL). The solution was dried over anhydrous MgSO₄. The product (0.250 g, 71% yield) was isolated as a white solid by chromatography (silica gel, 60% ethyl acetate in hexane).

### Examples 15-18

Compounds having formula (C), wherein R₁ and R₂ are shown in Table 2, were prepared using the method described in Example 14, starting with the corresponding isothiocyanate and amidine.

TABLE 2

| | | | Total |
|---|---|---|---|
| | | | yield |
| Ex. | R₁ | R₂ | (%) |
| 15 | phenyl | phenyl | 70 |
| 16 | phenyl | 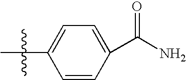 | 65 |
| 17 | 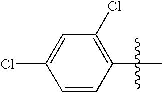 | phenyl | 54 |
| 18 | 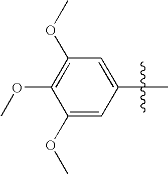 | t-butyl | 44 |

### Utility

The present invention enables an efficient and effective production of 1,2,4-triazole-3,5-diamine and/or 1,3,5-triazine-2,4-diamine derivatives, which are useful as pharmaceutical agents and/or as components of pharmaceutical agents, particularly inhibitors of IMPDH, anti-hypertensive agents, and neurokinin receptor antagonists, from readily-available starting compounds. This process provides a convenient synthesis of the target compound from a substituted isothiocyanate, hydrogencyanamide salt, a substituted hydrazine and/or a substituted amidine in moderate to good yield. The tri-nitrogen containing heteroaryl-diamine derivatives of formula (I), 1,2,4-triazole-3,5-diamines of formula (A) and/or (B) and the 1,3,5-triazine-2,4-diamines of formula (C), are expected to have activity as inhibitors of IMPDH. For example, the compound of Example 13 was shown to have an IC₅₀ value of 150 nM in an assay against the IMPDH II enzyme. Additionally, tri-nitrogen containing heteroaryl-diamine derivatives of formula (I), 1,2,4-triazole-3,5-diamines of formula (A) and/or (B) and 1,3,5-triazine-2,4-diamines of formula (C) may be readily coupled to a pharmacological core component to provide pharmaceutically active agents, such as, for examples, the compounds described in WO 00/25780, Dunstan et al., Tetrahedron Lett., Vol. 39, (1998), at pp. 7983-7986, U.S. Pat. No. 6,436,928, U.S. patent application No. 20020038030, U.S. Pat. No. 6,172,077 B1, U.S. Pat. No. 5,232,938, U.S. Pat. No. 4,569,933, WO 01/74806A1, WO 01/56987 A1, and EP 1107962 A1, incorporated herein by reference.

The compounds and pharmaceutical agents produced according to the inventive process may be used to treat inflammation, particularly inflammation characterized by the activation of T and/or B cells. The compounds and pharmaceutical agents thus produced can be immunomodulators and have multiple effects on cells of the immune system.

Compounds and pharmaceutical agents produced according to the inventive process will be useful in treating consequences of many diseases associated with chronic and acute inflammation and immune-modulation. Such diseases include, but are not limited to, inflammatory bowel disease, irritable bowel syndrome, gall bladder disease, Crohn's disease, rheumatoid arthritis, osteoarthritis, osteoporosis, traumatic arthritis, rubella arthritis, muscle degeneration, pancreatis (acute or chronic), psoriasis, glomerulonephritis, serum sickness, lupus (systematic lupus erythematosis), urticaria, scleraclerma, schleroderma, chronic thyroiditis, Grave's disease, dermatitis (contact or atopic), dermatomyositis, alopecia, atopic eczemas, ichthyosis, fever, sepsis, migraine, cluster headaches, Alzheimer's Disease, Parkinson's disease, Creutzfeldt-Jacob disease, multiple sclerosis, tuberculosis, dementia, transplant or graft-host rejections (e.g., kidney, liver, heart, lung, pancreas, bone marrow, cornea, small bowel, skin allografts, skin homografts and heterografts, etc.); respiratory allergies and diseases including asthma, acute respiratory distress syndrome, hayfever, allergic rhinitis, and chronic obstructive pulmonary disease; inflammatory disorders of the central nervous system, including HIV, encephalitis, cerebral malaria, meningitis, and ataxia telangiectasis.

Neurokinin receptor antagonists prepared using the inventive processes are expected to be useful in the treatment or prevention of various disease states, for example, addictions such as alcohol dependence and psychoactive substance abuse; stress related disorders such as post traumatic stress disorder; obsessive/compulsive disorders; eating disorders such as bulimia, anorexia nervosa and binge eating disorders; mania; premenstrual syndrome; central nervous system conditions such as anxiety, general anxiety disorder, panic disorder, phobias, bipolar disorders, migraine, epilepsy, nociception, emesis, depression, psychosis, schizophrenia, Alzheimer's disease, AIDs related dementia and Towne's disease; gastrointestinal disorders such as Crohn's disease and colitis; nausea; bladder disorders; pain, and so forth.

Compounds and pharmaceutical agents producing according to the inventive processes may be incorporated into pharmaceutical compositions for administration to a patient. For examples, the compounds may be incorporated into compositions for oral administration including suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants such as those known in the art. The compounds and agents thus produced may be included in compositions for oral delivery by sublingual and/or buccal administration, e.g., with molded, compressed, or freeze-dried tablets. Exemplary compositions may include fast-dissolving diluents such as mannitol, lactose, sucrose, and/or cyclodextrins. Also included in such formulations may be high molecular weight excipients such as celluloses (AVICEL®) or polyethylene glycols (PEG); an excipient to aid mucosal adhesion such as hydroxypropyl cellulose (HPC), hydroxypropyl methyl cellulose (HPMC), sodium carboxymethyl cellulose (SCMC), and/or maleic anhydride copolymer (e.g., GANTREZ®); and agents to control release such as polyacrylic copolymer (e.g., CARBOPOL 934®). Lubricants, glidants, flavors, coloring agents and stabilizers may also be added for ease of fabrication and use.

Compounds and pharmaceutical agents produced according to the inventive processes also may be incorporated into pharmaceutical compositions for nasal aerosol or inhalation administration, for parenteral administration, or rectal administration. Compositions for nasal administration may include solutions which may contain, for example, benzyl alcohol or other suitable preservatives, absorption promoters to enhance absorption and/or bioavailability, and/or other solubilizing or dispersing agents such as those known in the art. Compositions for parenteral administration may include injectable solutions or suspensions which may contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides and fatty acids, including oleic acid. Compositions for rectal administration may include suppositories which may contain, for example, suitable non-irritating excipients, such as cocoa butter, synthetic glyceride esters or polyethylene glycols, which are solid at ordinary temperatures but liquefy and/or dissolve in the rectal cavity to release the drug.

The effective amount of a compound produced according to the inventive processes may be determined by one of ordinary skill in the art. The specific dose level and frequency of dosage for any particular subject may vary and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition. An exemplary effective amount of pharmaceutical agent produced according to the invention may be within the dosage range of about 0.1 to about 100 mg/kg, preferably about 0.2 to about 50 mg/kg and more preferably about 0.5 to about 25 mg/kg (or from about 1 to about 2500 mg, preferably from about 5 to about 2000 mg) on a regimen in single or 2 to 4 divided daily doses. Preferred subjects for treatment include animals, most preferably mammalian species such as humans, and domestic animals such as dogs, cats, horses, and the like.

What is claimed is:

1. A process of producing at least one 1,2,4-triazole-3,5-diamine derivative comprising reacting an isothiocyanate with a hydrogencyanamide salt to produce an N-cyanothiourea salt, then reacting the N-cyanothiourea salt with a hydrazine in the presence of a peptide-coupling reagent to provide the at least one 1,2,4-triazole-3,5-diamine derivative.

2. The process of claim 1, wherein the isothiocyanate and hydrogencyanamide salt are reacted in an organic solvent selected from one or more of an aprotic polar solvent, an ether solvent, an alcohol solvent, and a halogen-containing solvent.

3. The process of claim 1, wherein the hydrogencyanamide salt has a formula of M(NHCN)ₙ, wherein M is selected from Li, Na, K, Mg, Ca, and Ba, n is 1 or 2, and the peptide-coupling reagent is selected from 1,3-dicyclohexylcarbodiimide, 1-(3-dimethylaminopropyl)-3-ethylcarbodiimide hydrochloride, 1-benzotriazol-1-yloxy-bis(pyrrolidino)uronium hexafluorophosphate, 5-(1H-benzotriazol-1-yloxy)-3,4-dihydro-1-methyl 2H-pyrrolium hexachloroanitimonate, benzotriazol-1-yloxy-N,N-dimethylmethaniminium hexachloroantimonate, O-(7-azabenzotriazol-1-yl)-1,1,3,3-tetramethyluronium hexafluorophosphate, O-(7-azabenzotriazol-1-yl)-1,1,3,3-bis(tetramethylene)uronium hexafluorophosphate, O-(benzotriazol-1-yl)-1,1,3,3-tetramethyluronium hexafluorophosphate, O-(7-azabenzotriazol-1-yl)-1,1,3,3-bis(pentamethylene)uronium tetrafluoroborate, O-(7-azabenzotriazol-1-yl)-tris(dimethylamino)phosphonium hexafluorophosphate, benzotriazol-1-yl diethyl phosphate, benzotriazol-1-yloxy-tris (dimethylamino) phosphonium hexafluorophosphate, 7-azobenzotriazolyoxytris(pyrrolidino)phosphonium hexafluorophosphate, 1-benzotriazolyoxytris(pyrrolidino)phosphonium hexafluorophosphate, 2-(3,4-dihydro-4-oxo-1,2,3-benzotriazin-3-yl)-1,1,3,3-tetramethyluronium tetrafluoroborate, 2-(5-norbornene-2,3-dicarboximido)-1,1,3,3-tetramethyluronium tetrafluoroborate, 2-(2-oxo-1(2H)-pyridyl-1,1,3,3-tetramethyluronium tetrafluoroborate, 2-succinimido-1,1,3,3-tetramethyluronium tetrafluoroborate, 2-bromo-3-ethyl-4-methyl thiazolium tetrafluoroborate, bis(2-oxo-3-oxazolidinyl)phosphinic chloride, bromotris(dimethylamino)phosphonium hexafluorophosphate, bis(tetramethylenefluoroformamidinium) hexafluorophosphate, 2-chloro-1,3-dimethylimidazolidinium hexafluorophosphate, 3-(diethoxyphosphoryloxy)-1,2,3-benzotriazin-4(3H)-one, diphenylphosphinic chloride, 2-ethoxy-1-ethoxycarbonyl-1,2-dihydroquinoline, pentafluorophenyl diphenylphosphinate, S-(1-oxido-2-pyridinyl)-1,1,3,3-tetramethylthiouronium hexafluorophosphate-PF6, S-(1-oxido-2-pyridinyl)-1,1,3,3-tetramethylthiouronium tetrafluoroborate-BF4, bromotris(pyrrolydino)phophonium hexafluorophosphate, chlorotris(pyrrolydino)phophonium hexafluorophosphate, tetramethylfluoroformamidinium hexafluorophosphate, carbonyldiimidazole, 1-hydroxybenzotriazole, 1-hydroxy-7-azabenzotriazole, 3-hydroxy-3,4-dihydro-4-oxo-1,2,3-benzotriazine, hydroxysuccinimide and N-ethyl-5-phenylisoxazolium-3′-sulfonate.

4. The process of claim 1, wherein the process is performed in one pot.

5. A process of producing a 1,3,5-triazine-2,4-diamine derivative comprising reacting an isothiocyanate with a hydrogencyanamide salt to produce an N-cyanothiourea salt, then reacting the N-cyanothiourea salt with an amidine in the presence of a peptide-coupling reagent to provide the 1,3,5-triazine-2,4-diamine derivative.

6. The process of claim 5, wherein the isothiocyanate and hydrogencyanamide salt are reacted in an organic solvent selected from one or more of an aprotic polar solvent, an ether solvent, an alcohol solvent, and a halogen-containing solvent.

7. The process of claim 5, wherein the hydrogencyanamide salt has a formula of M(NHCN)ₙ, wherein M is selected from Li, Na, K, Mg, Ca, and Ba, n is 1 or 2, and the peptide-coupling reagent is selected from 1,3-dicyclohexylcarbodiimide, 1-(3-dimethylaminopropyl)-3-ethylcarbodiimide hydrochloride, 1-benzotriazol-1-yloxy-bis(pyrrolidino)uronium hexafluorophosphate, 5-(1H-benzotriazol-1-yloxy)-3,4-dihydro-1-methyl 2H-pyrrolium hexachloroanitimonate, benzotriazol-1-yloxy-N,N-dimethylmethaniminium hexachloroantimonate, O-(7-azabenzotriazol-1-yl)-1,1,3,3-tetramethyluronium hexafluorophosphate, O-(7-azabenzotriazol-1-yl)-1,1,3,3-bis(tetramethylene)uronium hexafluorophosphate, O-(benzotriazol-1-yl)-1,1,3,3-tetramethyluronium hexafluorophosphate, O-(7-azabenzotriazol-1-yl)-1,1,3,3-bis(pentamethylene)uronium tetrafluoroborate, O-(7-azabenzotriazol-1-yl)-tris(dimethylamino)phosphonium hexafluorophosphate, benzotriazol-1-yl diethyl phosphate, benzotriazol-1-yloxy-tris(dimethylamino)phosphonium hexafluorophosphate, 7-azobenzotriazolyoxytris(pyrrolidino)phosphonium hexafluorophosphate, 1-benzotriazolyoxytris(pyrrolidino)phosphonium hexafluorophosphate, 2-(3,4-dihydro-4-oxo-1,2,3-benzotriazin-3-yl)-1,1,3,3-tetramethyluronium tetrafluoroborate, 2-(5-norbornene-2,3-dicarboximido)-1,1,3,3-tetramethyluronium tetrafluoroborate, 2-(2-oxo-1(2H)-pyridyl-1,1,3,3-tetramethyluronium tetrafluoroborate, 2-succinimido-1,1,3,3-tetramethyluronium tetrafluoroborate, 2-bromo-3-ethyl-4-methyl thiazolium tetrafluoroborate, bis(2-oxo-3-oxazolidinyl)phosphinic chloride, bromotris(dimethylamino)phosphonium hexafluorophosphate, bis(tetramethylenefluoroformamidinium)hexafluorophosphate, 2-chloro-1,3-dimethylimidazolidinium hexafluorophosphate, 3-(diethoxyphosphoryloxy)-1,2,3-benzotriazin-4(3H)-one, diphenylphosphinic chloride, 2-ethoxy-1-ethoxycarbonyl-1,2-dihydroquinoline, pentafluorophenyl diphenylphosphinate, S-(1-oxido-2-pyridinyl)-1,1,3,3-tetramethylthiouronium hexafluorophosphate-PF6, S-(1-oxido-2-pyridinyl)-1,1,3,3-tetramethylthiouronium tetrafluoroborate-BF4, bromotris(pyrrolydino)phophonium hexafluorophosphate, chlorotris(pyrrolydino)phophonium hexafluorophosphate, tetramethylfluoroformamidinium hexafluorophosphate, carbonyldiimidazole, 1-hydroxybenzotriazole, 1-hydroxy-7-azabenzotriazole, 3-hydroxy-3,4-dihydro-4-oxo-1,2,3-benzotriazine, hydroxysuccinimide and N-ethyl-5-phenylisoxazolium-3′-sulfonate.

8. The process of claim 5, wherein the process is performed in one pot.

9. A process for producing at least one tri-nitrogen containing heteroaryl-diamine derivative of formula (I): 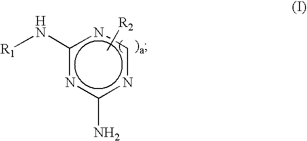
in which a is 0 or 1 and R₁ and R₂ are independently selected from alkyl, substituted alkyl, cycloalkyl, heterocyclo, aryl, and heteroaryl,
which comprises:
(a) reacting an isothiocyanate (R₁NCS) with a hydrogencyanamide salt in an organic solvent to produce an N-cyanothiourea salt;
(b) reacting the N-cyanothiourea salt with (i) a hydrazine (R₂NHNH₂), an amidine (R₂C(NH)NH₂), a salt of a hydrazine with one or more equivalents of a base, or a salt of an amidine with one or more equivalents of a base, and (ii) in the presence of a peptide-coupling reagent, to afford the tri-nitrogen containing heteroaryl-diamine derivatives of the formula (I).

10. The process of claim 9, wherein step (a) and step (b) are performed in one pot.

11. The process of claim 9, wherein:
R₁ is selected from alkyl, C₃₋₇cycloalkyl, phenyl or benzyl optionally substituted with one to four R₄;
R₂ is selected from alkyl, C₃₋₇cycloalkyl, and phenyl optionally substituted with one to two R₅; and
R₄ and R₅ are independently selected from halogen, lower alkoxy, trifluoromethyl, trifluoromethoxy, cyano, lower alkyl, and optionally substituted phenyl.

12. The process of claim 9, wherein:
(i) the hydrogencyanamide salt is sodium hydrogencyanamide;
(ii) the organic solvent is selected from one or more of an aprotic polar solvent, an ether solvent, an alcohol solvent, and a halogen-containing solvent; and
(iii) the peptide-coupling reagent is selected from 1,3-dicyclohexylcarbodiimide, 1-(3-dimethylaminopropyl)-3-ethylcarbodiimide hydrochloride, 1-benzotriazol-1-yloxy-bis(pyrrolidino)uronium hexafluorophosphate, 5-(1H-benzotriazol-1-yloxy)-3,4-dihydro-1-methyl 2H-pyrrolium hexachloroanitimonate, benzotriazol-1-yloxy-N,N-dimethylmethaniminium hexachloroantimonate, O-(7-azabenzotriazol-1-yl)-1,1,3,3-tetramethyluronium hexafluorophosphate, O-(7-azabenzotriazol-1-yl)-1,1,3,3-bis(tetramethylene)uronium hexafluorophosphate, O-(benzotriazol-1-yl)-1,1,3,3-tetramethyluronium hexafluorophosphate, O-(7-azabenzotriazol-1-yl)-1,1,3,3-bis(pentamethylene)uronium tetrafluoroborate, O-(7-azabenzotriazol-1-yl)-tris(dimethylamino)phosphonium hexafluorophosphate, benzotriazol-1-yl diethyl phosphate, benzotriazol-1-yloxy-tris (dimethylamino) phosphonium hexafluorophosphate, 7-azobenzotriazolyoxytris(pyrrolidino)phosphonium hexafluorophosphate, 1-benzotriazolyoxytris(pyrrolidino)phosphonium hexafluorophosphate, 2-(3,4-dihydro-4-oxo-1,2,3-benzotriazin-3-yl)-1,1,3,3-tetramethyluronium tetrafluoroborate, 2-(5-norbornene-2,3-dicarboximido)-1,1,3,3-tetramethyluronium tetrafluoroborate, 2-(2-oxo-1(2H)-pyridyl-1,1,3,3-tetramethyluronium tetrafluoroborate, 2-succinimido-1,1,3,3-tetramethyluronium tetrafluoroborate, 2-bromo-3-ethyl-4-methyl thiazolium tetrafluoroborate, bis(2-oxo-3-oxazolidinyl)phosphinic chloride, bromotris(dimethylamino)phosphonium hexafluorophosphate, bis(tetramethylenefluoroformamidinium)hexafluorophosphate, 2-chloro-1,3-dimethylimidazolidinium hexafluorophosphate, 3-(diethoxyphosphoryloxy)-1,2,3-benzotriazin-4(3H)-one, diphenylphosphinic chloride, 2-ethoxy-1-ethoxycarbonyl-1,2-dihydroquinoline, pentafluorophenyl diphenylphosphinate, S-(1-oxido-2-pyridinyl)-1,1,3,3-tetramethylthiouronium hexafluorophosphate-PF6, S-(1-oxido-2-pyridinyl)-1,1,3,3-tetramethylthiouronium tetrafluoroborate-BF4, bromotris(pyrrolydino)phophonium hexafluorophosphate, chlorotris(pyrrolydino)phophonium hexafluorophosphate, tetramethylfluoroformamidinium hexafluorophosphate, carbonyldiimidazole, 1-hydroxybenzotriazole, 1-hydroxy-7-azabenzotriazole, 3-hydroxy-3,4-dihydro4-oxo-1,2,3-benzotriazine, hydroxysuccinimide and N-ethyl-5-phenylisoxazolium-3′-sulfonate.

13. The process of claim 9, wherein step (a) comprises adding the hydrogencyanamide salt to the isothiocyanate in an anhydrous organic solvent at ambient temperature, stirring the first mixture at a first elevated temperature for a first time period, the first time period being sufficient to cause the hydrogencyanamide salt to react with the isothiocyanate, and then cooling the reaction mixture to ambient temperature.

14. The process of claim 9, wherein step (b) comprises adding the hydrazine, amidine, salt of a hydrazine with one or more equivalents of a base, or salt of an amidine with one or more equivalents of a base and the peptide-coupling reagent to the reaction mixture at ambient temperature and stirring the mixture at a second elevated temperature for a second time period, the second time period being sufficient to produce the at least one tri-nitrogen containing heteroaryl-diamine derivative of formula (I).

15. The process of claim 1, further comprising (i) coupling the at least one 1,2,4-triazole-3,5-diamine derivative to a pharmacological core component or (ii) converting the 1,3,5-triazine-2,4-diamine derivative to produce a pharmacologically-active agent.

16. A pharmaceutical product containing at least one 1,2,4-triazole-3,5-diamine derivative of a formula (A) and/or (B) produced according to the process of claim 1, wherein formula (A) and/or (B) are as follows:

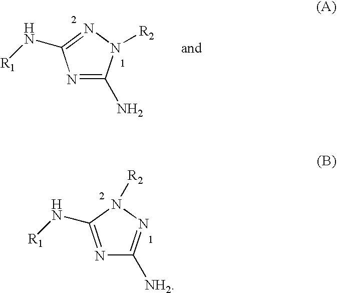

17. The process of claim 5, further comprising (i) coupling the 1,3,5-triazine-2,4-diamine derivative to a pharmacological core component or (ii) converting the 1,3,5-triazine-2,4-diamine derivative to produce a pharmacologically-active agent.

18. A pharmaceutical product containing a 1,3,5-triazine-2,4-diamine derivative of formula (C) produced according to the process of claim 5, wherein formula (C) is as follows:

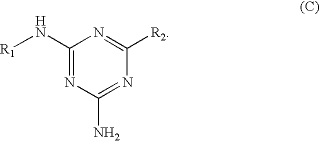

19. The process of claim 9, further comprising (i) coupling the at least one tri-nitrogen containing heteroaryl-diamine derivative or (ii) converting the at least one tri-nitrogen containing heteroaryl-diamine derivative to produce a pharmacologically-active agent.

20. A pharmaceutical product containing at least one tri-nitrogen containing heteroaryl-diamine derivative of formula (I) produced according to the process of claim 9, wherein formula (I) is as follows:

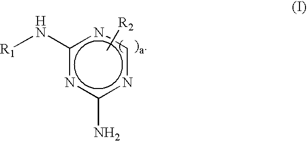

21. A process for making a pharmaceutical agent comprising:
providing an isothiocyanate having the formula (XNCS) wherein X comprises a pharmacological core component;
reacting said isothiocyanate with a hydrogencyanamide salt in an organic solvent to produce a reaction mixture;
reacting the reaction mixture with (i) a hydrazine, an amidine, a salt of a hydrazine with one or more equivalents of a base, or a salt of an amidine with one or more equivalents of a base, (ii) in the presence of a peptide-coupling reagent, to afford the pharmaceutical agent.
